(19) United States
(12) Patent Application Publication
(10) Pub. No.: US 2020/0170580 A1
(43) Pub. Date: Jun. 4, 2020

(54) METHODS FOR ASSESSING GRAFT FAILURE RISK

(71) Applicants: INSERM (INSTITUT NATIONAL DE LA SANTÉ ET DE LA RECHERCHE MÉDICALE), Paris (FR); UNIVERSITÉ DE LIMOGES, Limoges (FR); CENTRE HOSPITALIER RÉGIONAL UNIVERSITAIRE DE LIMOGES, Limoges (FR)

(72) Inventors: Aurélie PREMAUD, Limoges (FR); Annick ROUSSEAU, Limoges (FR); Marie ESSIG, Limoges (FR)

(21) Appl. No.: 16/615,905

(22) PCT Filed: May 23, 2018
(86) PCT No.: PCT/EP2018/063456

§ 371 Date: Nov. 22, 2019

(30) Foreign Application Priority Data
May 24, 2017 (EP) 17305611.0

(51) Int. Cl. A61B 5/00; G01N 33/70; G01N 33/68; G16H 50/30

(52) U.S. Cl. CPC A61B 5/7275; G01N 33/70; G01N 2333/70539; G16H 50/30; G01N 2800/245; G01N 33/6878

(57) ABSTRACT

The present invention relates to methods for assessing graft failure risk. Many predictive models of graft survival based on large panels of data collected exist but a limitation of these models is that they do not take into account the onset of adverse events over time, which modify graft outcome. The inventors developed a conditional and adjustable score, taking into account onset of emerging risks over time such as development of dnDSA, for prediction of graft failure (AdGFS) up to 10 years post-transplantation in 664 kidney transplant patients. AdGFS was externally validated and calibrated in 896 kidney transplant patients. In particular, the present invention relates to a method of assessing graft failure risk in a subject by measuring several factors: serum creatinine concentration, de novo donor-specific anti-HLA antibodies, pretransplant non donor-specific anti-HLA antibodies, acute rejection, age, proteinuria longitudinal serum creatinine cluster.

### FIELD OF THE INVENTION

The present invention relates to methods for assessing graft failure risk.

### BACKGROUND OF THE INVENTION

Scoring systems that predict survival outcome after kidney transplantation can help physicians improve risk stratification among recipients and make the best therapeutic decision for a patient who develops de novo donor-specific anti-human leucocyte antigen (HLA) antibody (DSA). Serum creatinine (Scr) and estimated glomerular filtration rate (GFR) are not sufficiently reliable predictors for long-term risk of graft loss or patient death (Kaplan B, Schold J, Meier-Kriesche H-U. Poor predictive value of serum creatinine for renal allograft loss. Am J Transplant Off J Am Soc Transplant Am Soc Transpl Surg. 2003; 3: 1560-1565). In the last decade, predictive models of graft survival based on large panels of data collected in the donor (Nyberg S L, Matas A J, Kremers W K, Thostenson J D, Larson T S, Prieto M, et al. Improved scoring system to assess adult donors for cadaver renal transplantation. Am J Transplant Off J Am Soc Transplant Am Soc Transpl Surg. 2003; 3: 715-721), in the recipient before transplantation (Brown T S, Elster E A, Stevens K, Graybill J C, Gillern S, Phinney S, et al. Bayesian modeling of pretransplant variables accurately predicts kidney graft survival. Am J Nephrol. 2012; 36: 561-569), and/or in the first year post-transplantation (Foucher Y, Daguin P, Akl A, Kessler M, Ladriere M, Legendre C, et al. A clinical scoring system highly predictive of long-term kidney graft survival. Kidney Int. 2010;78: 1288-1294) have been proposed. A limitation of these models is that they do not take into account the onset of adverse events over time, which modify graft outcome. In particular, these models never consider the impact of the development of de novo (dn)DSA beyond one year post-transplantation on graft outcome, although this has been demonstrated to be strongly associated with graft loss through antibody-mediated rejections (Hourmant M, Cesbron-Gautier A, Terasaki P I, Mizutani K, Moreau A, Meurette A, et al. Frequency and clinical implications of development of donor-specific and non-donor-specific HLA antibodies after kidney transplantation. J Am Soc Nephrol JASN. 2005; 16: 2804-2812). The previously proposed tools were globally validated in patient cohorts but they often lost their predictive power in small patient subgroups with specific risks of graft failure, i.e. the patients who need them most.

Development of a graft failure risk score is most often based on Cox's proportional hazards models (eventually with time-dependent covariates) to identify predictive risk factors (Foucher Y, Daguin P, Akl A, Kessler M, Ladriere M, Legendre C, et al. A clinical scoring system highly predictive of long-term kidney graft survival. Kidney Int. 2010; 78: 1288-1294) (Kasiske B L, Israni A K, Snyder J J, Skeans M A, Peng Y, Weinhandl E D. A simple tool to predict outcomes after kidney transplant. Am J Kidney Dis Off J Natl Kidney Found. 2010; 56: 947-960) (Shabir S, Halimi J-M, Cherukuri A, Ball S, Ferro C, Lipkin G, et al. Predicting 5-year risk of kidney transplant failure: a prediction instrument using data available at 1 year posttransplantation. Am J Kidney Dis Off J Natl Kidney Found. 2014; 63: 643-651). Random survival forest (RSF) modeling is an alternative non-parametric method based on an ensemble tree method for the analysis of right censored survival data (Ishwaran H, Kogalur U B, Blackstone E H, Lauer M S. Random survival forests. Ann Appl Stat. 2008; 2: 841-860). RSF was found able to identify complex interactions among multiple variables and performed better than traditional cox proportional hazard model (Miao F, Cai Y-P, Zhang Y-X, Li Y, Zhang Y-T. Risk Prediction of One-Year Mortality in Patients with Cardiac Arrhythmias Using Random Survival Forest. Comput Math Methods Med. 2015; 2015: 30325). Other advantages of RSF are (i) insensitivity to noise brought by missing values or error data and (ii) inclusion of an internal validation process. Thus, RSF has been used in several risk models in cardiology (Hsich E, Gorodeski E Z, Blackstone E H, Ishwaran H, Lauer M S. Identifying important risk factors for survival in patient with systolic heart failure using random survival forests. Circ Cardiovasc Qual Outcomes. 2011; 4: 39-45) and oncology (Ishwaran H, Blackstone E H, Apperson-Hansen C, Rice T W. A novel approach to cancer staging: application to esophageal cancer. Biostat Oxf Engl. 2009; 10: 603-620). A conditional scoring system may be more appropriate than the addition of weights as derived from Cox model if the impact of a risk factor is different, whether or not it is associated with other factors. Finally, a prognostic tool that can be updated with comorbidity onset may be more powerful (Sene M, Taylor J M, Dignam J J, Jacqmin-Gadda H, Proust-Lima C. Individualized dynamic prediction of prostate cancer recurrence with and without the initiation of a second treatment: Development and validation. Stat Methods Med Res. 2014).

### SUMMARY OF THE INVENTION

The present invention relates to methods for assessing graft failure risk. In particular, the invention is defined by the claims.

### DETAILED DESCRIPTION OF THE INVENTION

### Definitions

As used herein, the term “graft” refers to organs and/or tissues and/or cells which can be obtained from a first mammal (or donor) and transplanted into a second mammal (or recipient), preferably a human. The term “graft” encompasses, for example, skin, eye or portions of the eye (e.g., cornea, retina, lens), muscle, bone marrow or cellular components of the bone marrow (e.g., stem cells, progenitor cells), heart, lung, heartlung, liver, kidney, pancreas (e.g., islet cells, β-cells), parathyroid, bowel (e.g., colon, small intestine, duodenum), neuronal tissue, bone and vasculature (e.g., artery, vein). Preferably, a graft according to the invention is kidney.

As used herein, the term “acute rejection” or “graft rejection” is the rejection by the immune system of a tissue transplant when the transplanted tissue is immunologically foreign. It is possible to distinguish antibody mediated rejection (ABMR) and T-cell mediated rejection (TCMR). Acute cellular rejection is characterized by infiltration of the transplanted tissue by immune cells of the recipient, which carry out their effector function and destroy the transplanted tissue. ABMR is a pathological process that is associated with pathogenic donor specific anti-HLA antibodies (DSA).

As used herein, the term “graft failure” refers to loss of function in a transplanted organ or tissue. In kidney transplant patients, graft failure often means return to dialysis.

As used herein, the term “subject” denotes a mammal, such as a rodent, a feline, a canine, and a primate. Preferably, a subject according to the invention is a human. Preferably, a subject according to the invention is a recipient. In one embodiment, the subject is kidney transplant patient. As used herein, the term “kidney transplant patient” refers to a subject that has undergone kidney transplantation.

The term “donor” as used herein refers to the subject that provides the organ and/or tissue transplant or graft to be transplanted into the recipient and/or host.

The term “recipient” or “host” as used herein refers to any subject that receives an organ and/or tissue transplant or graft.

As used herein, the term “transplantation” refers the transfer of an organ and/or tissue from one human or non-human animal (i.e., a “donor”) to another human or non-human animal (i.e., a recipient).

As used herein, the term “predicting” refers to a probability or likelihood for a subject to develop an event. Preferably, the event is herein graft failure.

As used herein, the term “assessing” refers to the evaluation of the probability for a subject to develop graft failure.

As used herein, the term “dynamic prediction” refers to providing an assessment of probability or likelihood for a subject to develop graft failure, which may change over time.

As used herein, a predetermined reference can be relative to a number or value derived from population studies, obtained from the general population or from a selected population of subjects. Such predetermined reference values can be derived from statistical analyses and/or risk assessment data of populations obtained from mathematical algorithms and computed indices. The predetermined reference value can be a threshold value or a range. For example, the selected population may be comprised of apparently healthy transplanted patient, such as individuals who have not previously had any sign or symptoms indicating the outcome of a graft failure.

As used herein, the term “risk” refers to the probability that an event will occur over a specific time period, such as the onset of graft failure, and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a patient compared either to the absolute risks of low risk cohorts or an average population risk, which can vary by how clinical risk factors are assessed. According to the invention, there are four risk levels: low risk, intermediate risk, high risk or very high risk of graft failure. A risk factor is an individual factor able to increase the probability of graft dysfunction and/or graft failure.

As used herein, the term “creatinine” has its general meaning in the art and refers to 2-Amino-1-methyl-1H-imidazol-4-ol, a breakdown product of creatine phosphate in muscle. As used herein, the term “serum creatinine concentration” refers to the concentration of creatinine in the serum of said subject.

As used herein, the term “de novo donor-specific anti-HLA antibodies” has its general meaning in the art and refers to anti-human leucocyte antigen (HLA) antibodies which are donor specific and occur in the subject after the transplantation.

As used herein, the term “pretransplant non donor-specific anti-HLA antibodies” has its general meaning in the art and refers to anti-human leucocyte antigen (HLA) antibodies which are not donor specific and which are present in the subject before the transplantation.

As used herein, the term “age” refers to the period of a subject life, measured by years from birth.

As used herein, the term “score” refers to a piece of information, usually a number that conveys the result of the subject on a test. A risk scoring system separates a patient population into different risk groups; herein the process of risk stratification classifies the patients into very high-risk, high-risk, intermediate-risk and low-risk groups. In the context of the present invention, the score refers to a conditional and adjustable score over time for prediction of graft failure (AdGFS).

As used herein, the term “parameter” refers to any characteristic tested when carrying out the method according to the invention. In the context of the present invention, a parameter may be for instance the presence or the absence of de novo donor-specific anti-HLA antibodies, the presence or the absence of pretransplant non donor-specific anti-HLA antibodies, the presence or the absence of acute rejection, the age of the donor, the proteinuria or the longitudinal serum creatinine cluster.

As used herein, the term “parameter variable” refers to a value (a number for instance) associated to a parameter. In the context of the present invention, for the parameter “proteinuria” for instance, the parameter variables may be 0.18 g/L, 0.19 g/L or any proteinuria concentration measured in the subject. In the context of the present invention, for the parameter “the presence or the absence of de novo donor-specific anti-HLA antibodies” for instance, the parameter variables may be the presence of de novo donor-specific anti-HLA antibodies or the absence of de novo donor-specific anti-HLA antibodies in the subject. The presence of anti-HLA antibodies refers to anti-HLA antibodies mean fluorescence intensity (MFI) higher than a predetermined cut-off value. Said predetermined cutoff value is used for determining positive detection of anti-HLA antibodies. In one embodiment, the predetermined cut-off value is equal to 1000 MFI.

As used herein, the term “weight” refers to a value assigned to each parameter variable. The weights were determined statistically using results of Random Survival Forest analysis and were adjusted by maximizing the area under the time-dependent receiver operating characteristic (ROC) curves for censored survival data at different times post-transplantation. The addition of the weights of parameter variables tested for a subject when carrying out the method of the present invention corresponds to the final score (conditional and adjustable score for prediction of graft failure (AdGFS) in the context of the invention). The final score permits the assessment of the graft failure risk of said tested subject. For example, FIG. 2 shows each weight assigned to each parameter variable: +0, +2, +4, +10.

As used herein, the term “proteinuria” refers to the presence of proteins in the urine in excess of normal levels.

As used herein, the term “longitudinal serum creatinine cluster” refers to homogeneous subgroups of trajectories of serum creatinine measured within the first year post-transplantation. Subjects classified in the same cluster have close time-trajectories (at each time point) with similar shapes. Clustering adds information to the use of single or repeated measurement(s) of biological or clinical markers. Herein, it revealed patient subgroups with homogenous serum creatinine time-profiles.

### Prediction Methods of the Invention

The inventors developed a conditional and adjustable score for prediction of graft failure (AdGFS) up to 10 years post-transplantation in 664 kidney transplant patients. AdGFS was externally validated and calibrated in 896 kidney transplant patients.

The final model included five baseline factors (pretransplant non donor-specific anti-HLA antibodies, donor age, serum creatinine measured at 1 year, longitudinal serum creatinine clusters during the first year, proteinuria measured at 1 year), and two predictors updated over time (onset of de novo donor-specific anti-HLA antibodies and first acute rejection). AdGFS was able to stratify patients into four risk-groups, at different post-transplantation times. It showed good discrimination (time-dependent ROC curve at ten years: 0.83 (CI95% 0.76-0.89)).

Thus, the inventors built (using RSF and conditional trees) and validated a new conditional risk-scoring system of graft failure up to ten years post-transplantation, taking into account onset of emerging risks over time such as development of dnDSA. Their score highlights the impact of renal function during the first year and the evolution of the risk of graft loss with the onset of dnDSA and acute rejection.

Accordingly, a first object of the present invention relates to a method of assessing graft failure risk over time, at different times from one to ten years after transplantation, in a subject having serum creatinine concentration lower than a predetermined low reference, said method comprising:
a) Analyzing the presence or the absence of de novo donor-specific anti-HLA antibodies;
b) Analyzing the presence or the absence of pretransplant non donor-specific anti-HLA antibodies when no de novo donor-specific anti-HLA antibodies were detected as positive at step a) or analyzing the presence or the absence of acute rejection when de novo donor-specific anti-HLA antibodies were detected as positive at step a);
c) Comparing the age of donor with a predetermined reference;
d) Assessing the short- and long-term graft failure risks by calculating a conditional and adjustable score for dynamic prediction of graft failure (AdGFS) by adding the predefined weights assigned to each parameters variables tested at steps a), b) and c);
e) Concluding that the subject has a low risk, an intermediate risk or a high risk of graft failure on each date of the calculation of the score.

In one embodiment, the method of the present invention comprises:
i) as long as no de novo donor-specific anti-HLA antibodies are detected as positive at step a), no pretransplant non donor-specific anti-HLA antibodies are detected at step b) and the age of the donor is lower than a predetermined reference at step c);
ii) it is concluded that said subject has a low risk of graft failure.

In one embodiment, the method of the present invention comprises:
i) as long as no de novo donor-specific anti-HLA antibodies are detected as positive at step a), no pretransplant non donor-specific anti-HLA antibodies are detected at step b) and the age of the donor is higher than a predetermined reference at step c);
ii) it is concluded that said subject has an intermediate risk of graft failure.

In one embodiment, the method of the present invention comprises:
i) as long as no de novo donor-specific anti-HLA antibodies are detected as positive at step a), pretransplant non donor-specific anti-HLA antibodies are detected at step b) and the age of the donor is lower than a predetermined reference at step c);
ii) it is concluded that said subject has an intermediate risk of graft failure.

In one embodiment, the method of the present invention comprises:
i) as long as no de novo donor-specific anti-HLA antibodies are detected as positive at step a), pretransplant non donor-specific anti-HLA antibodies are detected at step b) and the age of the donor is higher than a predetermined reference at step c);
ii) it is concluded that said subject has a high risk of graft failure.

In one embodiment, the method of the present invention comprises:
i) as soon as de novo donor-specific anti-HLA antibodies are detected as positive at step a), as long as no acute rejection has been detected at step b) and the age of the donor is lower than a predetermined reference at step c);
ii) it is concluded that said subject has an intermediate risk of graft failure.

In one embodiment, the method of the present invention comprises:
i) as soon as de novo donor-specific anti-HLA antibodies are detected as positive at step a), as long as no acute rejection has been detected at step b) and the age of the donor is higher than a predetermined reference at step c);
ii) it is concluded that said subject has a high risk of graft failure.

In one embodiment, the method of the present invention comprises:
i) as soon as de novo donor-specific anti-HLA antibodies are detected as positive at step a), when acute rejection has been detected at step b) and the age of the donor is lower than a predetermined reference at step c);
ii) it is concluded that said subject has a high risk of graft failure.

In one embodiment, the method of the present invention comprises:
i) as soon as de novo donor-specific anti-HLA antibodies are detected as positive at step a), when acute rejection has been detected at step b) and the age of the donor is higher than a predetermined reference at step c);
ii) it is concluded that said subject has a high risk of graft failure.

A second object of the present invention relates to a method of predicting graft failure risk over time, at different times from one to ten years after transplantation, in a subject having serum creatinine concentration higher than or equal to a predetermined low reference and lower than or equal to a predetermined high reference, said method comprising:
a) Measuring the proteinuria;
b) Identifying the first year longitudinal serum creatinine cluster of said subject when proteinuria measured is lower than a predetermined reference;
c) Comparing the age of the donor with a predetermined reference;
d) Assessing the graft failure risk by calculating a conditional and adjustable score for dynamic prediction of graft failure (AdGFS) by adding the predefined weights assigned to each parameters variables tested at steps a), b) and c);
e) Concluding that the subject has an intermediate risk, a high risk or a very high risk of graft failure on each date of the calculation of the score.

In one embodiment, the method of the present invention comprises:
i) when proteinuria measured at step a) is lower than a predetermined reference, said subject belongs to the longitudinal serum creatinine cluster B as identified in step b) and the age of the donor is lower than a predetermined reference at step c);
ii) it is concluded that said subject has an intermediate risk of graft failure.

In one embodiment, the method of the present invention comprises:
i) when proteinuria measured at step a) is lower than a predetermined reference, said subject belongs to the longitudinal serum creatinine cluster B as identified in step b) and the age of the donor is higher than a predetermined reference at step c);
ii) it is concluded that said subject has a high risk of graft failure.

In one embodiment, the method of the present invention comprises:
i) when proteinuria measured at step a) is lower than a predetermined reference, said subject belongs to the longitudinal serum creatinine cluster A or C as identified in step b) and the age of the donor is lower than a predetermined reference at step c);
ii) it is concluded that said subject has a high risk of graft failure.

In one embodiment, the method of the present invention comprises:
i) when proteinuria measured at step a) is lower than a predetermined reference, said subject belongs to the longitudinal serum creatinine cluster A or C as identified in step b) and the age of the donor is higher than a predetermined reference at step c);
ii) it is concluded that said subject has a high risk of graft failure.

In one embodiment, the method of the present invention comprises:
i) when proteinuria measured at step a) is higher than a predetermined reference and the age the donor is lower than a predetermined reference at step c);
ii) it is concluded that said subject has a high risk of graft failure.

In one embodiment, the method of the present invention comprises:
i) when proteinuria measured at step a) is higher than a predetermined reference and the age of the donor is higher than a predetermined reference at step c);
ii) it is concluded that said subject has a very high risk of graft failure.

A further object of the present invention relates to a method of assessing graft failure risk in a subject, said method comprising:
i) measuring serum creatinine concentration
ii) concluding that said subject has a very high risk of graft failure when serum creatinine concentration is higher than a predetermined high reference.

According to the invention, there are a serum creatinine concentration predetermined low reference and a serum creatinine concentration predetermined high reference. In the context of the invention, it is understood that the value of the predetermined low reference is inferior to the value of the predetermined high reference.

In one embodiment, serum creatinine concentration predetermined low reference is lower than or equal to 200 μM. In one embodiment, serum creatinine concentration predetermined low reference is equal to 150 μM.

In one embodiment, serum creatinine concentration predetermined high reference is comprised between 200 and 400 μM. In one embodiment, serum creatinine concentration predetermined high reference is equal to 272 μM.

In one embodiment, repeated measurements of serum creatinine concentration are performed during all the patient follow-up up to ten years post-transplantation. In one embodiment, serum creatinine concentration is measured within the first 12 months after transplantation.

In one embodiment, proteinuria predetermined reference is lower or equal to 0.275 g/L or 1 g/24 h. In one embodiment, proteinuria predetermined reference is equal to 0.275 g/L or 1 g/24 h.

In one embodiment, proteinuria is measured between transplantation and 24 months after transplantation. In one embodiment, proteinuria is measured 12 months after transplantation.

In one embodiment, the donor age predetermined reference is comprised between 45 and 70 years. In one embodiment, the donor age predetermined reference is equal to 60 years.

According to the invention, a subject may be classified in one of several longitudinal serum creatinine clusters. In one embodiment, there are three different longitudinal serum creatinine clusters A, B and C. In the context of the invention, cluster A refers to persistent low pattern with median serum creatinine of 105 μM (range: 38-206 μM). In the context of the invention, cluster B refers to intermediate pattern with median serum creatinine of 159 μM (range: 84-469 μM). In the context of the invention, cluster C refers to unstable high pattern with median serum creatinine of 248 μM (range: 85-900 μM).

According to the invention, there are several graft failure risk levels, represented by the score calculated with the methods of the present invention. In one embodiment, there are four risk levels: low risk, intermediate risk, high risk or very high risk of graft failure.

In one embodiment, a low graft failure risk is comprised between 4 and 8%.

In one embodiment, a low graft failure risk corresponds to about 6%.

In one embodiment, an intermediate graft failure risk is comprised between 17 and 29%.

In one embodiment, an intermediate graft failure risk corresponds to about 23%.

In one embodiment, a high graft failure risk is comprised between 35 and 55%.

In one embodiment, a high graft failure risk corresponds to about 45%.

In one embodiment, a very high graft failure risk is comprised 59 and 95%.

In one embodiment, a very high graft failure risk corresponds to about 77%.

Test to Determine Serum Creatinine Concentration:

There is many tests known by the skilled man to determine creatinine concentration. The tests the most used in routine measurement of creatinine concentration comprise the Jaffe's colorimetric method and the enzymatic method.

Test to Determine the Presence or the Absence of De Novo Donor-Specific Anti-HLA Antibodies:

There is several sensitive tests known by the skilled man to determine the presence of de novo donor-specific anti-HLA antibodies. For instance, an example of a test to determine the presence of de novo donor-specific anti-HLA antibodies comprises: screening of antibodies to HLA-A, HLA-B, HLA-C, HLA-DP, HLA-DQ and HLA-DR gene products using Luminex® solid-phase assay (one lambda Labscreen assay) on serum samples. In case of detection of an anti-HLA-antibody, the donor specificity of the antibody is determined by molecular DNA typing of the donor.

Test to Determine the Presence or the Absence of Pretransplant Non Donor-Specific Anti-HLA Antibodies:

There is several sensitive tests known by the skilled man to determine the presence or the absence of pretransplant non donor-specific anti-HLA antibodies. For instance, an example of a test to determine the presence or the absence of pretransplant non donor-specific anti-HLA antibodies comprises: screening of antibodies to HLA-A, HLA-B, HLA-C, HLA-DP, HLA-DQ and HLA-DR gene products using Luminex® solid-phase assay (one lambda Labscreen assay) on serum samples. In case of detection of an anti-HLA-antibody, the non donor specificity of the antibody is determined by molecular DNA typing of the donor.

Test to Determine the Presence or the Absence of Acute Rejection:

Graft biopsy was used to analyze histological graft lesions. The Banff classification is the recommended tool to classify and grade acute rejection.

Test to Determine the First Year Longitudinal Serum Creatinine Cluster:

There is many tests known by the skilled man to determine the first year longitudinal serum creatinine cluster. For instance, an example of a test to determine the first year longitudinal serum creatinine cluster comprises: clustering method based on k-means, specifically designed to analyse longitudinal data and implemented in the ‘kml’ R-package (version 1.1.3).

Test to Determine Proteinuria:

There is many tests known by the skilled man to determine proteinuria. For instance, an example of a test to determine proteinuria comprises the colorimetric method with pyrogallol red.

The method of the present invention allows analyzing simultaneously parameter variables which are associated to the progression of the disease while each isolated parameter is not reliable for assessing long-term risk of graft loss or patient death. The parameter variables most predictive of graft loss in the short- and long terms, i.e. the most relevant for clinical monitoring, are different upon the patients and the stage of their kidney disease. Therefore the present invention determines a patient risk-stratification based on a conditional schema. Indeed, a conditional scoring system is more appropriate than the addition of weights classically used if the impact of a risk factor is different on graft survival, whether or not it is associated with another factor. A dynamic prognostic tool that can be updated with each new biomarker measurement or comorbidity onset is the most powerful. In the literature, no scoring system for long-term kidney graft survival provided for recalculation of risk beyond 12 months after the transplantation and took into account onset of de novo donor-specific anti-HLA antibodies or acute rejection beyond one year after transplantation. In the state of the art, the other scoring systems for prediction of graft failure are static.

In one embodiment, the method of the present invention is used for selecting patients in the clinical trials.

While we are moving to the era of personalized therapies in kidney transplantation, prognostic tools are necessary for the optimal selection of patients in clinical trials and thereafter the choice of treated patients to test preventive therapeutic strategies of graft failure. Herein, the inventors have proposed a new method that integrates data collected during all the follow-up of the patient and clinical data to predict kidney survival in transplant patients, and enables to offer a personal approach to clinical decision making. To demonstrate significant effects of candidate molecules, future trials should focus on patients with poor renal prognoses, and the AdGFS score may be a valuable tool that could identify these patients. Indeed, patients from the high-risk group appear to meet this definition, as they have about a 45% risk of evolution to graft failure before ten years after transplantation. In sharp contrast, patients from the low-risk group, should not be exposed to the potential side effects of candidate molecules, because they have good renal prognoses.

### Prevention Methods of the Present Invention

A further object of the present invention relates a method of preventing graft failure in a subject in need thereof, said method comprising:

i) assessing the graft failure risk by performing the method according the invention;

ii) increasing immunosuppressive regimen when it is concluded that the subject has a low risk, an intermediate or high risk of graft failure before diagnosis of DSA.

Another object of the present invention relates a method of preventing graft failure in a subject in need thereof, said method comprising:

i) assessing the graft failure risk by performing the method according the invention;

ii) increasing immunosuppressive regimen in order to better control de novo DSA, when it is concluded that the subject initially classified in the low risk group has moved to the intermediate risk group of graft failure after diagnosis of de novo DSA.

iii) contributing to evaluate the balance benefit/risk of increasing immunosuppressive regimen to better control de novo DSA, with respect to comorbidities and risk of side effects of a high immunosuppression when it is concluded that the subject initially classified in the intermediate or high risk group has moved to the high risk or very high risk group of graft failure after diagnosis of de novo DSA.

The method of the present invention may be used for risk managing to personalize and optimize surveillance and treatments. While the decision to treat or not to treat for DSA (by increasing immunosuppressive regimen) will be relatively straight forward for patients in the low-risk category before diagnosis of DSA, different factors may influence the clinical decision making for the other risk-groups. It will be understood that the total daily usage of the compounds and compositions of the present invention will be decided by the attending physician within the scope of sound medical judgment. The decision to treat or not to treat events such as onset of de novo DSA may be greatly help by the calculation of AdGFS. The specific therapeutic strategy for any particular subject will depend upon a variety of factors including risk-group of AdGFS, acute rejection episode(s), graft function (assessed with serum creatinine level, proteinuria) and comorbidities and like factors well known in the medical arts. For example, in patients with a short-term high risk (very high- and high-risk groups) of graft failure, specific medical strategy linked with onset of dnDSA might be personalized regarding the comorbidities of the patient and the balance between the probability of maintaining a functioning graft and the side effects associated to the treatments. The calculation of AdGFS score may contribute to evaluate the balance benefit/risk of increasing immunosuppressive regimen. It is known within the skill of the art that the onset of de novo DSA increases only moderately the graft failure risk in patient who did not experience acute rejection. Consequently, in such patients classified in the intermediate-risk group, in presence of at-risk clinical context, comorbidities such as cancer, difficulties linked to patient's tolerance of the molecule and the quality of the patient's life, a more intensive surveillance of rejections without specific individual adjustment of immunosuppressive regimen for DSA may be recommended. Nevertheless surveillance includes a close monitoring of the immunosuppressive drug exposure to avoid suboptimal exposure.

As used herein, the term “preventing” refers to the reduction in the risk of acquiring or developing a given condition.

As used herein, the term “immunosuppressive regimen” refers to the administration of immunosuppressive drugs to a patient in need thereof.

As used herein, the term “immunosuppressive drug” refers to any substance capable of producing an immunosuppressive effect, e.g., the prevention or diminution of the immune response.

In a particular embodiment, the immunosuppressive drug is selected from the group consisting of antithymocyte globulin (ATG), interleukin (IL)-2 Receptor Antagonists (Basiliximab and Daclizumab), alemtuzumab (Campath-1H), muromonab-CD3 (OKT3), azathioprine (AZA), glucocorticosteroids, calcineurin Inhibitors (Cyclosporine (CsA) and Tacrolimus (Tac)), mycophenolate mofetil (MMF) and Enteric-Coated Mycophenolate Sodium (EC-MPS), sirolimus, everolimus (RAD), belatacept, leflunomide, rituximab, bortezomib, eculizumab, alefacept, siplizumab (MEDI-507), sotrastaurin (AEB-071), janus Kinase (JAK)3 Inhibitor (CP-690550), voclosporin (ISA247) or TOL101.

### Devices of the Present Invention

A further object of the present invention relates to a computer program containing a set of instructions characteristic of implementation of the method of the present invention.

As used herein, the term “computer” refers to a machine having a processor, a memory, and an operating system, capable of interaction with an user or other computer, and shall include without limitation desktop computers, notebook computers, personal digital assistants (PDAs), servers, handheld computers, and similar devices.

As used herein, the term “computer program” refers to is a collection of instructions that performs a specific task when executed by a computer.

A further aspect of the present invention relates to an application program including means for implementing the method of the present invention. In one embodiment, the application program of the present invention is a smartphone application.

As used herein, the term “application program” refers to executable code such as a .exe file, Java applet or servlet, interpreted script, etc.

The invention will be further illustrated by the following figures and examples. However, these examples and figures should not be interpreted in any way as limiting the scope of the present invention.

### FIGURES

FIG. 1: Conditional inference tree applied for graft survival with predicted Kaplan-Meier curves in the terminal nodes. The tree was obtained using recursive partitioning for censored response in a conditional inference framework implemented in ‘party’ R-package.

FIG. 2: Scoring system for computing AdGFS values. ScrM12=serum creatinine at 12 months post-transplantation. ProtM12=proteinuria at 12 months post-transplantation. Scr=serum creatinine. dnDSA=de novo donor-specific anti-HLA antibodies. NDSA=non donor-specific anti-HLA antibodies.

FIG. 3: Comparison of Kaplan-Meier graft survival curves for the four risk groups namely low-, intermediate-, high-, and very high- risk of graft loss in the development dataset (solid lines) and in the external validation dataset (dashed lines). Patients were partitioned according to the calculated score value: low risk (0), intermediate risk (2 or 4), high risk (6 or 8), and very high risk (10 or 12). Graft survival in the development and validation datasets did not differ within each of the four risk groups.

### EXAMPLE

Material & Methods

This study adheres to the Declaration of Istanbul.

Database

Of the 819 transplantations performed at the University Hospital of Limoges (France) between december 1984 and december 2011, 664 were included in the primary cohort (development database). All 664 transplants studied came from heart-beating deceased donors and had a follow-up of at least one year after transplantation. The maintenance immunosuppressive regimen consisted mainly of one calcineurin inhibitor (cyclosporine or, since 2001, tacrolimus) associated with azathioprine (until 1996) or mycophenolate mofetil (after 1996) and corticosteroids (generally stopped between 3 and 6 months post-transplantation). All patients received induction therapy. Patient outcome was known for each patient at the date of the last follow-up. Death was considered as a censored event when the recipient died with a functioning graft. When graft function was not known on the exact date of death, the date of the last biological assessment before death was then considered as the censoring time. Usually, graft function was recorded a few days before death. When patients died because of graft loss, death was considered as a graft failure.

Donor, recipient and graft characteristics were collected from the CRISTAL register (from the French public agency “Agence de la Biomedecine”). Samples for immunological analysis were available in the local biobank, declared at the Ministry of Health (N° DC-2010-1074). The study database was approved by the French Informatics and Liberty National Commission (CNIL, registration number 1795293).

Anti-HLA Antibodies Screening

Anti-HLA-A, -B, -C, -DP, -DQ, -DR antibodies were screened and identified using Luminex® solid-phase assay (One Lambda LABScreen assays) in samples collected before transplantation and routinely after transplantation (three, six, twelve months, once every year thereafter, and whenever clinically indicated). Results were expressed as median fluorescence intensity (MFI). MFI>1000 was considered positive. All sera tested using the Complement Dependent Cytotoxicity method prior to the availability of Luminex® technology in our center (2007), were re-analyzed using Luminex®. As DQ, DP and C HLA typing was not previously systematically performed in our center, a molecular DNA typing of donor and recipient was performed in case of detection by Luminex® of an anti-HLA-C, -DQ or -DP antibody during the survey. This procedure allowed to determine the specificity (donor-specific or non donor-specific) of the anti-HLA antibody and to avoid bias in the determination of DSA. DSA diagnosis prior to renal transplantation was an exclusion criterion for transplantation in our center. Patients in whom the Luminex® reanalysis identified presence of DSA before transplantation (n=13) were excluded from the database studied.

Cluster Analysis of Serum Creatinine over the First Year Post-Transplantation

Homogeneous subgroups of trajectories of serum creatinine measured within the first year post-transplantation were identified by a clustering method based on k-means, specifically designed to analyze longitudinal data and implemented in the ‘kml’ R-package (version 1.1.3) (Genolini C, Falissard B. KmL: a package to cluster longitudinal data. Comput Methods Programs Biomed. 2011; 104: e112-121). This method does not require any assumption regarding the shape of the serum creatinine-time curves, contrary to model-based methods which fit the trajectories with a specific model (e.g. linear, polynomial or exponential). The optimal number of clusters was selected using the statistical criterion proposed by Calinski and Harabasz (Calinski T, Harabasz J. A dendrite method for cluster analysis. Commun Stat. 1974; 3: 1-27).

Identification of Factors Predictive of Graft Survival

The impact of the following variables was investigated on graft survival: (i) donor characteristics (age, cause of death—cardiac, stroke or traumatic injuries—); (ii) recipient demographic variables (age at time of transplantation, gender); (iii) transplantation characteristics [time period of transplantation (i.e. 1984-1993, 1994-2003 or 2004-2011), cold ischemia time, previous kidney transplantation(s)]; (iv) immunological variables (HLA-A, HLA-B and HLA-DR mismatches, pre-transplant anti-HLA antibodies, source of anti-HLA alloimmunization (i.e. previous transplantation, pregnancy, blood transfusion), occurrence of de novo donor-specific and/or non-donor-specific anti-HLA antibodies (dnDSA and dnNDSA, respectively) with the date of the first diagnosis; (v) biological variables [repeated measurements of serum creatinine (μM) over the first year post-transplantation, proteinuria (g/L) at one year post-transplantation]; (vi) clinical variables (initial renal disease, date of first acute rejection diagnosis, date of return to dialysis, date of end of follow-up); and (vii) immunosuppressive drugs administered. Patient ethnicity was not recorded since it is not authorized by French law.

RSF analysis was performed to select and rank the most predictive covariates of graft failure using the date of transplantation as time origin (Ishwaran H, Kogalur U B, Blackstone E H, Lauer M S. Random survival forests. Ann Appl Stat. 2008; 2: 841-860). RSF was implemented in the ‘randomForestSRC’ R-package (version 2.0.0). Briefly, a RSF was generated by creating 1000 trees, each tree built on a randomly selected bootstrap sample (using 63% of the original data) using a randomly selected subset of covariates. Each bootstrap sample excluded, on average, 37% of the data, which were reserved for a test set called “out-of-bag” data (OOB). RSF evaluated the change in prediction error attributable to each covariate. The prediction error (i.e. the percentage of patients misclassified) was assessed with the Harrell's concordance index (Harrell's c-index) using OOB data (Harrell F E, Califf R M, Pryor D B, Lee K L, Rosati R A. Evaluating the yield of medical tests. JAMA. 1982; 247: 2543-2546). The c-index was computed using an OOB set constructed with the 1000 OOB datasets provided by the 1000 bootstrap samples used in growing the forest. The OOB prediction error is defined as 1 minus Harrell's c-index. The prediction error ranges between 0 and 1, where a value of 0.5 corresponds to a prediction no better than random guessing and a value of 0 reflects perfect accuracy. The parameter “nsplit” used to specify random splitting was fixed at 3. The predictive performance of the studied variables was evaluated by their “variable importance” (VIMP), calculated by RSF. VIMP measures the change in prediction error for a forest grown with or without this variable.

Variables selection was successively done by (1) fitting data by RSF and ranking all available variables and (2) iteratively fitting RSF by removing at each iteration a variable from the bottom of the positive variable importance ranking list. The minimal combination of variables leading to the smallest “out-of-bag” prediction error rate, assessed by the Harrell's c-index, was selected.

A conditional survival tree (Hothorn T, Hornik K, Zeileis A. Unbiased recursive partitioning: a conditional inference framework. J Comput Graph Stat. 2006; 15: 651-674) was subsequently drawn from the whole original dataset, using the most predictive variables selected from RSF ['party' (version 1.0-21) R-package].

Prediction of Graft Failure

Score calculations were derived from both the VIMP sourced from the final RSF model and the conditional survival tree. The weight of each variable (i.e. each risk factor) was based on the ratio between its VIMP and the VIMP of the last predictive variable retained. A same value of weight was allocated for variables split at the same tree-depth in the conditional survival tree. The weighted risk score was calculated by adding the weights of the different risk factors within each branch of the conditional survival tree. This strategy led to a score for each patient subgroup identified at each terminal node of the conditional survival tree. Time-dependent receiver operating characteristic (ROC) curves with area under the curve (AUC) for censored survival data were used to evaluate the discrimination of the developed score. Additional weights were attributed for variables not selected in the conditional survival tree but highly associated with graft survival in the RSF analysis, provided their inclusion improved the ROC AUC. The weight of a factor could be increased if it allowed maximization of the ROC AUC at ten years post-transplantation. The predictive performance of the developed score was evaluated by time-dependent sensitivity, specificity, positive predictive value (PPV) and negative predictive value (NPV) with their standard error, all estimated at several cutpoints, i.e. for different threshold score values and for different times after transplantation. Therefore, ‘timeROC’ (version 0.2) R package was employed using the Kaplan-Meier estimator of the censoring distribution. Baseline (i.e. including variables available at one year post-transplantation) and adjusted (i.e. adding variables collected after one year post-transplantation) scores were also compared using time-dependent ROC AUC.

External Validation

External validation of the developed score was performed in patients transplanted between 2002 and 2010 in two independent French transplantation centers (CHU Tours n=706; CHU Poitiers n=190). As in the development cohort, patients with pre-transplant DSA were excluded. All anti-HLA antibodies screenings were performed using Luminex®. The validation database (Astre database) was approved by the CNIL (Authorization number DR-2012-518).

Validation procedure included: recalculation of the Scr clusters considering the external database only, calculation of the individual scores using the developed scoring system, determination of the time-dependent ROC AUC at ten years post-transplantation and calibration based on Hosmer-Lemeshow goodness-of-fit test adapted for survival data (Leteurtre S, Martinot A, Duhamel A, Proulx F, Grandbastien B, Cotting J, et al. Validation of the paediatric logistic organ dysfunction (PELOD) score: prospective, observational, multicentre study. Lancet Lond Engl. 2003; 362: 192-197). The calibration evaluation consisted in comparing numbers of patients with graft failure expected and observed in the validation cohort using the calculation of the numbers of events based on Kaplan-Meier survival estimates which was by proposed by D'Agostino-Nam (D'Agostino R B, Nam B-H. Evaluation of the performance of survival analysis models: discrimination and calibration measure. Handbook of Statistics, Survival Methods. 2004. pp. 1-25). In a first step, the number of graft failures observed in the validation cohort in different time-intervals ([0-2[,[2-4[, [4-6[, [6-8[, [8-10] years after transplantation) were calculated for each risk group as the product nᵢ(1-KMᵢ(t)) where KMᵢ is the Kaplan-Meier survival estimate at a fixed time t for groupᵢ and nᵢ the number of observations in groupᵢ. The survival probabilities expected in the validation cohort were calculated using the Kaplan-Meier estimates obtained in the development cohort. With this test, the p value has to be higher than 0.05.

Statistical Analyses

Comparison between categorical data was done using the Pearson chi-square test or the exact Fisher test. Normally distributed data were analyzed by Anova and the parametric t-test, whereas nonparametric tests (Kruskall-Wallis and Mann-Whitney tests respectively) were used otherwise. Kaplan-Meier analysis was used to assess graft survival (graft loss, i.e. return to dialysis). Graft survival in different patient subgroups was compared using the log rank test.

Statistical analyses were performed with MedCalc for Windows, version 14.10.2. (MedCalc Software, Ostend, Belgium) and R version 2.15.1 (www.R-project.org). The R packages are freely available through the Comprehensive R Archive Network distribution system (http://cran.r-proj ect.org).

Results

Development Database

The characteristics of the studied kidney transplants are listed in Table 1.

TABLE 1
Kidney transplant characteristics of the development and validation databases.

| | Development | | | | | |
|---|---|---|---|---|---|---|
| | Database | | Validation Database (n = 896) | | | |
| | (n = 664) | | Tours | | Poitiers | |
| Total number of transplants | 664 | | 706 | | 190 | |
| Duration of follow-up (years) | 6.4 | (±3.3) | 7.4 | (±2.3) | 7.9 | (±1.2) |
| Functional renal grafts at 10 | 202 | (30.4%) | 171 | (24.2%) | 31 | (16.3%) |
| years post-transplantation | | | | | | |
| Recipient gender (M/F) | 405/259 | | — | | — | |
| Recipient age (years) | 49 | (±14) | — | | — | |
| Donor age (years) | 44 | (±16) | 51 | (±17) | 48 | (±15) |
| HLA A Mismatch | 1.2 | (±0.7) | — | | — | |
| HLA B Mismatch | 1.5 | (±0.6) | — | | — | |
| HLA DR Mismatch | 1.2 | (±0.7) | — | | — | |
| First transplantation | 608 | (91.6%) | — | | — | |
| Pretransplant NDSA | 105 | (15.8%) | 151 | (21.4%) | 34 | (17.9%) |
| Serum creatinine at M12 (μM) | 139 | (±71) | 136 | (±49) | 131 | (±40) |
| Proteinuria at M12 (g/L) | 0.18 | (±0.50) | | | 0.19 | (±0.45) |
| Proteinuria at M12(g/24 h) | | | 0.63 | (±1.96) | 0.37 | (±0.80) |
| Return to dialysis | 69 | (10.4%) | 116 | (16.4%) | 22 | (11.6%) |
| Death with a functional | 60 | (9.0%) | — | | — | |
| graft (censured data) | | | | | | |
| de novo NDSA | 142 | (21.3%) | — | | — | |
| Median (range) time to | 3.02 | (0.02-10) | | | | |
| onset (years) | | | | | | |
| de novo DSA | 62 | (9.3%) | 113 | (16%) | 41 | (21.6%) |
| Median (range) time to | 3.92 | (0.02-9.83) | 2.94 | (0.79-5.04) | 3.12 | (1.95-5.1) |
| onset (years) | | | | | | |
| Patients with onset of | 11 | (17.7%) | 35 | (31.0%) | 7 | (16.7%) |
| dnDSA in the first year | | | | | | |
| after transplantation | | | | | | |
| First acute rejection | 137 | (20.6%) | 219 | (31%) | 48 | (25.3%) |
| episode | | | | | | |
| Median (range) time | 0.26 | (0.01-9.27) | 0.15 | (0.04-0.35) | 0.25 | (0.03-0.40) |
| to onset (years) | | | | | | |

Data are n (%), mean (±SD) or median (range)
DSA = donor-specific anti-HLA antibodies.
NDSA = non-donor-specific anti-HLA antibodies.
M12 = month 12 post-transplantation.
— data not collected

During the whole study period, 137 patients have been treated for a first acute rejection among them 122 (89%) were biopsy proven. One hundred nine first rejections occurred during the first year post-transplantation. Borderline rejection was evidenced in 36 patients and T-Cell mediated rejection (TCMR) in 105 patients, Antibody-mediated rejection (ABMR) in 14 patients and mixed (ABMR+TCMR) in three patients. Only two patients displayed ABMR criteria on a biopsy done before the definition of ABMR in the Banff classification.

During follow-up, dnDSA were present in 62 patients. The median time to dnDSA diagnosis was significantly lower in patients who exhibited pretransplant NDSA than in patients who did not (1.42 vs 4.87 years, p=0.0012). Sixty-four percent of patients with dnDSA (n=39) had class II antigens, 34% (n=21) had class I and 2% (n=2) had both class I and II antigens. Nearly all patients who developed dnDSA after transplantation had previously (n=19) or concomitantly (n=36) developed dnNDSA. Except for one patient who presented dnDSA transiently (i.e., detected at 1.6 years after transplantation and absent at subsequent screenings), DSA remained persistent at all screenings following the first detection. Thirteen patients with dnDSA returned to dialysis, including six within the year following the diagnosis of dnDSA (median 1.04 years, range: 0.03-4.46). Eleven out the 17 patients with ABMR on histology had developed dnDSA.

Scr profiles over the first year post-transplantation were best partitioned in three clusters (data not shown). Graft survival after transplantation was significantly different in these three subgroups (p<0.0001) (data not shown). The percentage of donors over 60 years of age increased from cluster A to C (29 [7.7%], 57 [23.4%], and 19 [44.2%], respectively, p<0.0001). The mean cold ischemia time was significantly higher in cluster C than in clusters A and B, p=0.034). No cold ischemia time lower than 12 hours was observed in cluster C.

### Identification of Factors Predictive of Graft Survival After the First Year Post-Transplantation

The best model was obtained using the log rank splitting rule with 1000 trees with a Harrell's Concordance error rate of 21% (standard deviation 0.2%) (data not shoxn). This final model included five baseline variables (pretransplant NDSA, donor age, Scr measured at 12 months post-transplantation (ScrM12), Scr clusters, proteinuria measured at M12 (ProtM12)), and two predictors which could be updated during the follow-up of the graft (onset of dnDSA and first acute rejection whatever the time of onset after transplantation).

The partial plots of graft survival, predicted in the RSF analysis using the retained continuous variables (after adjusting for all other predictors) showed decreased survival when donor age exceeds 60 years, and very steep survival curves when ScrM12>150 μM, so that small increments in ScrM12 would result in large survival declines (data not shown).

Adjustable Graft Failure Score (AdGFS) for Prediction of Graft Survival

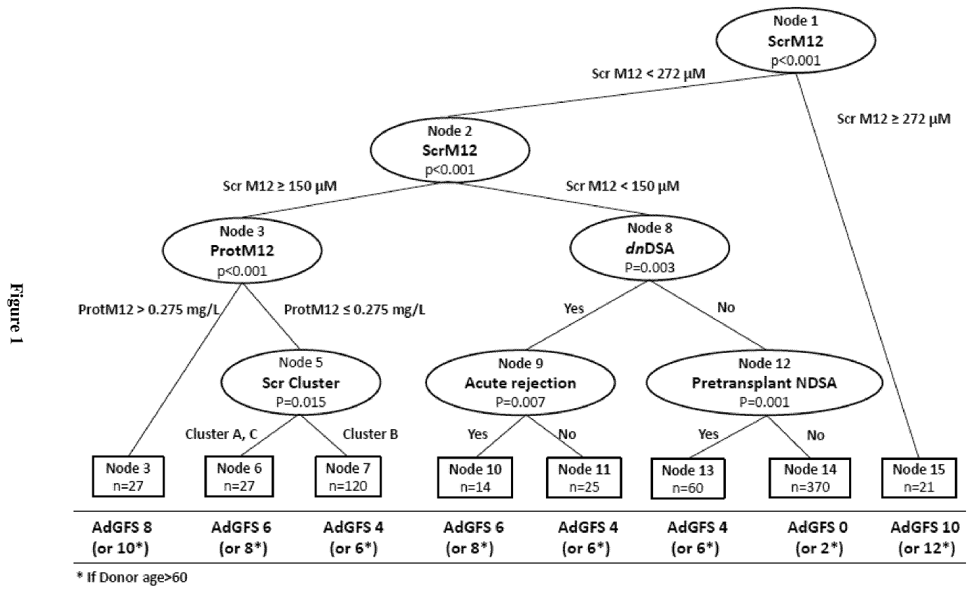

A scoring system was constructed using conditional survival tree analysis, with nodes corresponding to the variables selected in the final RSF model. The tree identified height terminal nodes, corresponding to height patient subgroups (FIG. 1). The hierarchical order of the variables in predicting graft survival provided by the conditional survival tree was in accordance with the variable ranks obtained by RSF analysis.

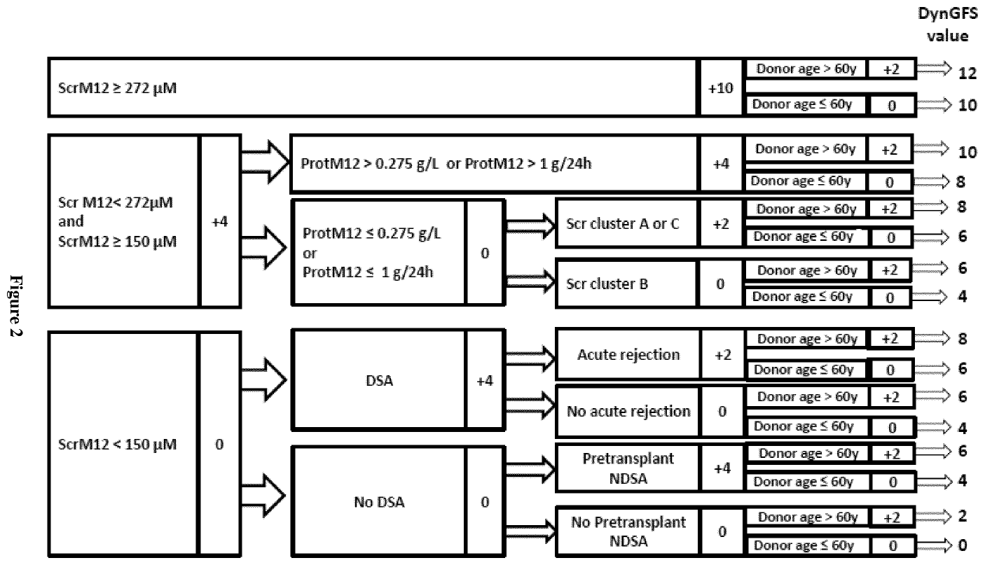

Our scoring system, named AdGFS (Adjustable Graft Failure Score), is shown in FIG. 2. AdGFS outperformed the baseline score including predictors available at one year after transplantation (time-dependent ROC AUC at ten years: 0.83 (CI95% 0.76-0.89) vs 0.75 (CI95% 0.68-0.82), p=0.0075). Taking into account onset of dnDSA and first acute rejection developed over time, after one year post-transplantation improved survival prediction beyond 5 years post-transplantation (p=0.0244).

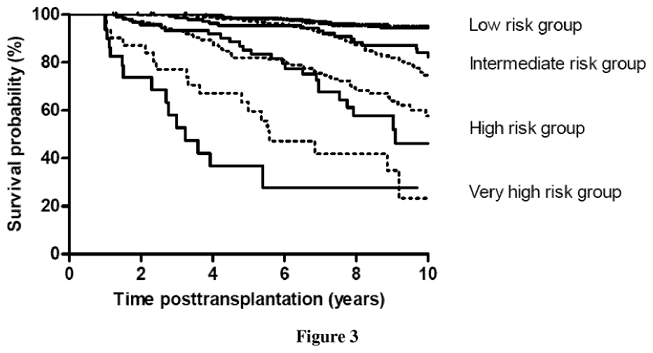

AdGFS values are reported for each patient subgroup in FIG. 1. Table 2 presents, for the different cutpoints of AdGFS values, the performance characteristics of graft survival prediction at different post-transplantation times. For example, a patient with low score (AdGFS=2) has a probability of graft survival up to 10 years post-transplantation of approximately 94.5% (NPV). Onset dnDSA during the follow-up increased the score value (adjusted score=6) and led to a probability of graft loss of 64.9% at 8 years and 83.6% at 10 years post-transplantation (PPV) (Table 2). Probabilities of graft survival lower than 20% (PPV>80%) at ten years post-transplantation were obtained for score values of 6 and more. Risk groups were defined according to the AdGFS value: low risk (0), intermediate risk (2-4), high risk (6-8), and very high risk (10-12). Ten years graft survival was significantly different between these four risk groups (p<0.0001) (FIG. 3).

TABLE 2
Performance characteristics of adjustable graft failure score (AdGFS)
for cutpoints 0, 2, 4, 6, 8, 10 and for different times over 10 years post-transplantation.

| | Number | Number | | | | | | | | | |
|---|---|---|---|---|---|---|---|---|---|---|---|
| Cutoff | of | of | Censored post- | | | | | | | | |
| point | positive | negative | transplantation | Se | | Sp | | PPV | | NPV | |
| (c) | tests (>c) | tests (≤c) | time (years) | (se_Se) % | | (se_Sp) % | | (se_PPV) % | | (se_NPV) % | |
| 0 | 292 | 365 | 2 | 100 | (0) | 59.7 | (2.1) | 4.3 | (1.3) | 100 | (0) |
| | 299 | 358 | 4 | 95.7 | (4.2) | 60.9 | (2.3) | 11.1 | (2.1) | 99.6 | (0.4) |
| | 303 | 354 | 6 | 85.7 | (5.9) | 66.2 | (2.6) | 17.9 | (2.9) | 98.1 | (0.8) |
| | 309 | 348 | 8 | 80.7 | (5.5) | 69.2 | (2.9) | 29.1 | (3.8) | 95.8 | (1.4) |
| | 314 | 343 | 10 | 79.4 | (5.4) | 72.5 | (3.2) | 35.9 | (4.5) | 94.8 | (1.5) |
| 2 | 264 | 393 | 2 | 100 | (0) | 63.2 | (2.1) | 4.6 | (1.4) | 100 | (0) |
| | 271 | 386 | 4 | 91.9 | (5.5) | 64.6 | (2.3) | 11.7 | (2.3) | 99.3 | (0.5) |
| | 275 | 382 | 6 | 83.3 | (6.2) | 68.5 | (2.5) | 18.5 | (3.0) | 97.9 | (0.9) |
| | 282 | 375 | 8 | 79.3 | (5.6) | 69.9 | (2.9) | 29.2 | (3.9) | 95.5 | (1.4) |
| | 288 | 369 | 10 | 78.3 | (5.4) | 73.1 | (3.2) | 36.0 | (4.5) | 94.5 | (1.5) |
| 4 | 120 | 537 | 2 | 100 | (0) | 85.4 | (1.5) | 11.0 | (3.2) | 100 | (0) |
| | 122 | 535 | 4 | 75.7 | (8.6) | 87.0 | (1.6) | 23.0 | (4.6) | 98.5 | (0.6) |
| | 125 | 532 | 6 | 70.6 | (7.4) | 91.0 | (1.6) | 40.2 | (6.2) | 97.3 | (0.8) |
| | 130 | 527 | 8 | 58.5 | (6.7) | 95.0 | (1.4) | 64.9 | (7.4) | 93.6 | (1.4) |
| | 134 | 523 | 10 | 53.7 | (6.5) | 97.7 | (1.0) | 83.6 | (7.2) | 91.6 | (1.6) |
| 6 | 62 | 595 | 2 | 90.9 | (8.7) | 93.3 | (1.1) | 19.8 | (5.7) | 99.8 | (0.2) |
| | 62 | 595 | 4 | 68.2 | (9.2) | 94.3 | (1.1) | 37.9 | (7.2) | 98.3 | (0.6) |
| | 62 | 595 | 6 | 56.6 | (8.0) | 96.0 | (1.1) | 55.2 | (8.4) | 96.2 | (1.0) |
| | 62 | 595 | 8 | 39.4 | (6.4) | 97.7 | (1.0) | 73.0 | (9.0) | 91.1 | (1.6) |
| | 62 | 595 | 10 | 33.1 | (5.7) | 98.4 | (0.9) | 80.7 | (9.5) | 88.4 | (1.9) |
| 8 | 31 | 626 | 2 | 62.8 | (14.7) | 97.5 | (0.7) | 31.1 | (10.0) | 99.3 | (0.4) |
| | 31 | 626 | 4 | 50.4 | (9.7) | 98.4 | (0.6) | 62.6 | (10.9) | 97.4 | (0.7) |
| | 31 | 626 | 6 | 34.2 | (7.5) | 99.1 | (0.5) | 77.6 | (11.0) | 94.6 | (1.1) |
| | 31 | 626 | 8 | 19.9 | (4.9) | 99.6 | (0.4) | 89.1 | (10.0) | 88.8 | (1.7) |
| | 31 | 626 | 10 | 16.7 | (4.2) | 100.0 | (0.0) | 100 | (0) | 86.1 | (1.9) |
| 10 | 9 | 648 | 2 | 17.6 | (11.4) | 99.1 | (0.4) | 26.2 | (16.2) | 98.5 | (0.5) |
| | 9 | 648 | 4 | 26.5 | (8.7) | 100 | (0) | 100 | (0) | 96.3 | (0.8) |
| | 9 | 648 | 6 | 16.3 | (5.8) | 100 | (0) | 100 | (0) | 93.3 | (1.2) |
| | 9 | 648 | 8 | 9.5 | (3.5) | 100 | (0) | 100 | (0) | 87.5 | (1.7) |
| | 9 | 648 | 10 | 7.9 | (3.0) | 100 | (0) | 100 | (0) | 84.9 | (1.9) |

Time post-transplantation was defined as the duration between the date of transplantation and the time point where graft failure prediction was made. The test was considered as positive when AdGFS score >cutpoint and negative when score was ≤cutpoint. Time dependent sensitivity (Se), Specificity (Sp) Positive Predictive Value (PPV) and Negative Predictive Value (NPV) were computed with standard error (se) at the six given cutpoints: 0 and 2, 4, 6, 8, 10 for different censored post-transplantation times. AdGFS could be calculated in 657 patients, 7 patients were secondarily excluded due to missing data.

External Validation of AdGFS

Table 1 reports the characteristics of the patients. Graft survival within each risk group was similar in the development and external validation datasets (FIG. 3). The accuracy of the score at predicting graft failure remained high in the validation dataset, with a time-dependent ROC AUC of 0.79 (CI 95% 0.74-0.84) at ten years after transplantation. Results of calibration evaluation of AdGFS in the external dataset were good: observed numbers of patients with graft failure were close to the expected numbers using the AdGFS risk groups ((χ₂=2.39, p=0.30) (Table 3).

TABLE 3
Goodness-of-fit test for external validation of the AdGFS score.

| | Number of patients | | Number of patients | |
|---|---|---|---|---|
| | with graft failure | | without graft failure | |
| Risk group | Observed | Expected | Observed | Expected |
| Low (0) | 14 | 14.9 | 314 | 313.1 |
| Intermediate (2 or 4) | 47 | 53.8 | 286 | 279.2 |
| High (6 or 8) | 57 | 58.3 | 146 | 144.7 |
| Very high (10 or 12) | 18 | 14.8 | 14 | 17.2 |

Data refers to the number of patients. Chi-squared = 2.39 (p = 0.30) with 2 degrees of freedom. The number of patients with graft failure expected in the validation cohort for the four different risk groups was calculated using the Kaplan-Meier survival estimates obtained in the development cohort.

Discussion

In the present work, we developed and externally validated a conditional and adjustable predictive score (named AdGFS) of long-term kidney graft failure including pre-transplantation, early post-transplantation predictors and two factors collected all along the patients' follow-up: onset of dnDSA and first acute rejection episodes. All the items included in the score are available everywhere in the day-to-day clinical surveillance of the patients. This score can be calculated from one year post-transplantation and updated all along the evolution of the graft depending on the occurrence of dnDSA and acute rejection. The calibration and discrimination of this score were good in large cohorts of patients treated with the current standard of care.

All previously published scores are computed using only individual factors known before the end of the first year post-transplantation. They are never updated, even if the patient's prognosis is altered. The performance of these scores is usually evaluated with respect to shorter term graft survival and at a single time point. In this study, we used the non-parametric RSF method which has several advantages compared to regression approaches among which it does not test the goodness of fit of data to a hypothesis, but seeks a model that explains the data.

The present study confirmed the deleterious role of donor age and its link with Scr (Nyberg S L, Matas A J, Kremers W K, Thostenson J D, Larson T S, Prieto M, et al. Improved scoring system to assess adult donors for cadaver renal transplantation. Am J Transplant Off J Am Soc Transplant Am Soc Transpl Surg. 2003; 3: 715-721). Donor age above 60 years was retained in different donor quality scoring systems and was also associated with graft outcome after acute ABMR (Loupy A, Lefaucheur C, Vernerey D, Chang J, Hidalgo L G, Beuscart T, et al. Molecular microscope strategy to improve risk stratification in early antibody-mediated kidney allograft rejection. J Am Soc Nephrol JASN. 2014;25: 2267-2277). In the present study, two other baseline predictors were identified: Scr cluster and pretransplant NDSA. Longitudinal Scr clusters, assessing the Scr time-profiles along the first year, have never been used before in predictive model of graft failure. Clustering adds information to the use of single or repeated measurement(s) of biological or clinical markers. Herein, it revealed patient subgroups with homogenous Scr time-profiles. This approach is in line with FDA guidance to better differentiate phenotypes of patients (http://www.fda.gov/downloads/Drugs/GuidanceComplianceregulatoryInformation/Guidances/UCM458485.pdf). For future studies, we propose a graphical tool dedicated to allocating new patients in the clusters (data not shown).

No previously proposed score takes into account onset of dnDSA beyond one year post-transplantation and their impact on graft survival. Our study, finding a cumulative incidence of 9.3% of dnDSA and a 24% rate of graft failure at 3 years after the onset of dnDSA, is in accordance with previous studies showing a 5-year post-transplantation cumulative incidence of dnDSA from 5.5 to 20%, a 7 to 9% risk of graft failure in the first year after the occurrence of dnDSA, and up to 24% of patients with chronic ABMR and renal failure within 3 years post-DSA.

AdGFS is the first score to include new-onset dnDSA to predict graft survival. The inclusion of dnDSA requires an adjustable approach since they may appear at any time. AdGFS can be updated during patient follow-up in case of dnDSA or acute rejection. DnDSA's pathogenicity depends on their association with acute rejection, as previously found by Cooper and colleagues (Cooper J E, Gralla J, Cagle L, Goldberg R, Chan L, Wiseman A C. Inferior kidney allograft outcomes in patients with de novo donor-specific antibodies are due to acute rejection episodes. Transplantation. 2011; 91: 1103-1109). Taking into account dnDSA improved survival prediction beyond 5 years post-transplantation in accordance with published works highlighting that graft loss attributable to dnDSA occurs several years after their onset (Everly M J, Rebellato L M, Haisch C E, Ozawa M, Parker K, Briley K P, et al. Incidence and impact of de novo donor-specific alloantibody in primary renal allografts. Transplantation. 2013; 95: 410-417).

Other factors classically reported to be associated with graft failure, such as HLA mismatches, cold ischemia, recipient gender, and immunosuppressive treatments, were not retained in the score because they did not allow a decrease in the error rate in the RSF analysis, and they did not improve the time-dependent ROC AUC. This was explained by their significant association with the retained variables (e.g increased cold ischemia time was associated with Scr clusters).

Contrary to published scores, AdGFS predicted graft failure at different post-transplantation times up to ten years and stratified the patients into four risk groups. Kasiske and colleagues (Kasiske B L, Israni A K, Snyder J J, Skeans M A, Peng Y, Weinhandl E D. A simple tool to predict outcomes after kidney transplant. Am J Kidney Dis Off J Natl Kidney Found. 2010; 56: 947-960) evaluated only the 5 year risk of graft failure and the discriminatory ability of their scores remained modest as highlighted by the authors. In the Kidney Transplant Failure Score, graft failure was evaluated at 8 years post-transplantation and patients were stratified into only two groups (Foucher Y, Daguin P, Aid A, Kessler M, Ladriere M, Legendre C, et al. A clinical scoring system highly predictive of long-term kidney graft survival. Kidney Int. 2010; 78: 1288-1294). The good results of our external validation in a population different with regards to time of transplantation and standard-of-care supported the robustness of AdGFS.

Assessment of the individual patient's risk of transplant failure throughout the time after transplantation may be a decisive tool to select the optimal care strategy for the patient. For instance, in the high risk group, specific treatments for dnDSA might be questionable regarding the balance between the probability of maintaining a functioning graft and the side effects associated to these treatments.

In conclusion, we propose an adjustable score for risk stratification of graft failure at different post-transplantation times. AdGFS showed good discrimination and could be more useful than scores ignoring onset of dnDSA, for decisions regarding more or less intensive surveillance and treatment of the patients.

### REFERENCES

Throughout this application, various references describe the state of the art to which this invention pertains. The disclosures of these references are hereby incorporated by reference into the present disclosure.

1. A method of assessing graft failure risk over time, at different times from one to ten years after transplantation, in a subject having serum creatinine concentration lower than a predetermined low reference, said method comprising:
a) analyzing the presence or the absence of de novo donor-specific anti-HLA antibodies;
b) analyzing the presence or the absence of pretransplant non donor-specific anti-HLA antibodies when no de novo donor-specific anti-HLA antibodies were detected as positive at step a) or analyzing the presence or the absence of acute rejection when de novo donor-specific anti-HLA antibodies were detected as positive at step a);
c) comparing the age of donor with a predetermined reference;
d) assessing the short- and long-term graft failure risks by calculating a conditional and adjustable score for dynamic prediction of graft failure (AdGFS) by adding the predefined weights assigned to each parameters variables tested at steps a), b) and c);
e) concluding that the subject has a low risk, an intermediate risk or a high risk of graft failure on each date of the calculation of the score.

2. The method of claim 1 wherein:
i) as long as no de novo donor-specific anti-HLA antibodies are detected as positive at step a), no pretransplant non donor-specific anti-HLA antibodies are detected at step b) and the age of the donor is lower than a predetermined reference at step c);
ii) it is concluded that said subject has a low risk of graft failure.

3. The method of claim 1 wherein:
i) as long as no de novo donor-specific anti-HLA antibodies are detected as positive at step a), no pretransplant non donor-specific anti-HLA antibodies are detected at step b) and the age of the donor is higher than a predetermined reference at step c);
ii) it is concluded that said subject has an intermediate risk of graft failure.

4. The method of claim 1 wherein:
i) as long as no de novo donor-specific anti-HLA antibodies are detected as positive at step a), pretransplant non donor-specific anti-HLA antibodies are detected at step b) and the age of the donor is lower than a predetermined reference at step c);
ii) it is concluded that said subject has an intermediate risk of graft failure.

5. The method of claim 1 wherein:
i) as long as no de novo donor-specific anti-HLA antibodies are detected as positive at step a), pretransplant non donor-specific anti-HLA antibodies are detected at step b) and the age of the donor is higher than a predetermined reference at step c);
ii) it is concluded that said subject has a high risk of graft failure.

6. The method of claim 1 wherein:
i) as soon as de novo donor-specific anti-HLA antibodies are detected as positive at step a), as long as no acute rejection has been detected at step b) and the age of the donor is lower than a predetermined reference at step c);
ii) it is concluded that said subject has an intermediate risk of graft failure.

7. The method of claim 1 wherein:
i) as soon as de novo donor-specific anti-HLA antibodies are detected as positive at step a), as long as no acute rejection has been detected at step b) and the age of the donor is higher than a predetermined reference at step c);
ii) it is concluded that said subject has a high risk of graft failure.

8. The method of claim 1 wherein:
i) as soon as de novo donor-specific anti-HLA antibodies are detected as positive at step a), when acute rejection has been detected at step b) and the age of the donor is lower than a predetermined reference at step c);
ii) it is concluded that said subject has a high risk of graft failure.

9. The method of claim 1 wherein:
i) as soon as de novo donor-specific anti-HLA antibodies are detected as positive at step a), when acute rejection has been detected at step b) and the age of the donor is higher than a predetermined reference at step c);
ii) it is concluded that said subject has a high risk of graft failure.

10. A method of assessing graft failure risk over time, at different times from one to ten years after transplantation, in a subject having serum creatinine concentration higher than or equal to a predetermined low reference and lower than or equal to a predetermined high reference, said method comprising:
a) measuring the proteinuria;
b) identifying the first year longitudinal serum creatinine cluster of said subject when proteinuria measured is lower than a predetermined reference;
c) comparing the age of the donor with a predetermined reference;
d) assessing the graft failure risk by calculating a conditional and adjustable score for dynamic prediction of graft failure (AdGFS) by adding the predefined weights assigned to each parameters variables tested at steps a), b) and c);
e) concluding that the subject has an intermediate risk, a high risk or a very high risk of graft failure on each date of the calculation of the score.

11. The method of claim 10 wherein:
i) when proteinuria measured at step a) is lower than a predetermined reference, said subject belongs to the longitudinal serum creatinine cluster B as identified in step b) and the age of the donor is lower than a predetermined reference at step c);
ii) it is concluded that said subject has an intermediate risk of graft failure.

12. The method of claim 10 wherein:
i) when proteinuria measured at step a) is lower than a predetermined reference, said subject belongs to the longitudinal serum creatinine cluster B as identified in step b) and the age of the donor is higher than a predetermined reference at step c);
ii) it is concluded that said subject has a high risk of graft failure.

13. The method of claim 10 wherein:
i) when proteinuria measured at step a) is lower than a predetermined reference, said subject belongs to the longitudinal serum creatinine cluster A or C as identified in step b) and the age of the donor is lower than a predetermined reference at step c);
ii) it is concluded that said subject has a high risk of graft failure.

14. The method of claim 10 wherein:
i) when proteinuria measured at step a) is lower than a predetermined reference, said subject belongs to the longitudinal serum creatinine cluster A or C as identified in step b) and the age of the donor is higher than a predetermined reference at step c);
ii) it is concluded that said subject has a high risk of graft failure.

15. The method of claim 10 wherein:
i) when proteinuria measured at step a) is higher than a predetermined reference and the age the donor is lower than a predetermined reference at step c);
ii) it is concluded that said subject has a high risk of graft failure.

16. The method of claim 10 wherein:
i) when proteinuria measured at step a) is higher than a predetermined reference and the age of the donor is higher than a predetermined reference at step c);
ii) it is concluded that said subject has a very high risk of graft failure.

17. A method of assessing graft failure risk in a subject, said method comprising:
i) measuring serum creatinine concentration
ii) concluding that said subject has a very high risk of graft failure when serum creatinine concentration is higher than a predetermined high reference.

18. A method of preventing graft failure in a subject in need thereof, said method comprising:
i) assessing the graft failure risk by performing the method according to claim 1, and
ii) increasing an immunosuppressive regimen when it is concluded that the subject has a low risk, an intermediate or high risk of graft failure before diagnosis of DSA.

19. An application program including means for implementing the method according to claim 1.

20. The method of claim 18, wherein the immunosuppressive regimen comprises administering one or more immunosuppressive drugs selected from the group consisting of antithymocyte globulin (ATG), an interleukin (IL)-2 receptor antagonist, alemtuzumab (Campath-1H), muromonab-CD3 (OKT3), azathioprine (AZA), a glucocorticosteroid, a calcineurin inhibitors, mycophenolate mofetil (MMF), enteric-coated mycophenolate sodium (EC-MPS), sirolimus, everolimus (RAD), belatacept, leflunomide, rituximab, bortezomib, eculizumab, alefacept, siplizumab (MEDI-507), sotrastaurin (AEB-071), a janus kinase (JAK)3 inhibitor, voclosporin (ISA247) and TOL101.
